(19) United States
(12) Patent Application Publication
(10) Pub. No.: US 2010/0130613 A1
(43) Pub. Date: May 27, 2010

(54) ADMINISTRATION OF ADAPALENE FOR MODULATING THE EXPRESSION OF CD1d OR IL-10

(75) Inventor: Brigitte DRENO, Nantes Cedex 01 (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, Biot (FR)

(21) Appl. No.: 12/468,528

(22) Filed: May 19, 2009

(30) Foreign Application Priority Data
Nov. 19, 2007 (FR) PCT/FR2007/052364

(51) Int. Cl. A61K 31/192; A61P 29/00

(52) U.S. Cl. 514/569

(57) ABSTRACT

Adapalene or pharmaceutically acceptable salt thereof is administered to an individual in need of such treatment for modulating the expression of CD1d or IL-10 in an inflammatory acneic lesion.

### CROSS-REFERENCE TO PRIORITY/PROVISIONAL APPLICATIONS

This application claims priority under 35 U.S.C. §119 of U.S. Provisional Application No. 60/859,968, filed Nov. 20, 2006, and is a continuation/national phase of PCT/FR 2007/052364, filed Nov. 19, 2007 and designating the United States (published in the French language on May 29, 2008 as WO 2008/062132 A1; the title and abstract were also published in English), each hereby expressly incorporated by reference in its entirety and each assigned to the assignee hereof.

### BACKGROUND OF THE INVENTION

1. Technical Field of the Invention

The present invention relates to the administration of adapalene for modulating the expression of CD1d or IL-10, in particular in patients suffering from acne.

2. Description of Background and/or Related and/or Prior Art

Acne is a chronic disease associated with inflammation of the pilosebaceous follicle, under hormonal control, and which involves three well-defined stages (Cunliffe W. J. et al., Br. J. Dermatol., 2000: 142: 1084-1091; Gollnick H. P. et al., J. Dermatol., 1991: 18: 489-499).

The first stage, which generally begins at puberty, corresponds to the stimulation of production by the sebaceous glands, inducing hyperseborrhoea. The second stage corresponds to the formation of microcomedones considered to be the first elementary lesion of acne caused by anomalies in the proliferation, adhesion and differentiation of keratinocytes in the lower part of the sebaceous canal of the hair follicle.

The third stage corresponds to the formation of inflammatory acneic lesions in which Propionibacterium acnes (P. acnes), an anaerobic bacterium, plays an essential role.

The treatments employed against acne are intended to prevent the development of bacteria with antiseptics such as benzoyl peroxide or antibiotics, or to reduce sebum secretion (retinoids).

Employed only in the case of severe acne, the oral treatments can be broken down into three categories: antibiotics (tetracycline and erythromycin), retinoids (especially represented by isotretinoin) and hormone treatments combining an oestrogen and a progestogen.

### SUMMARY OF THE INVENTION

It has now surprisingly been demonstrated that adapalene behaves like a modulator of the cytokines of the skin and makes it possible, in particular, to increase both the innate immune response and the acquired immune response of the skin via its action on 2 particular cytokines, CD1d and IL-10.

Indeed, the properties of this active ingredient have now been further investigated to improve the therapeutic efficacy thereof, and the mechanisms of action of adapalene have now been demonstrated in the treatment of acne.

The present invention thus features administration of adapalene, or of a pharmaceutically acceptable salt thereof, for increasing the expression of CD1d in an inflammatory skin lesion, in particular in an inflammatory acneic lesion. In particular, this invention features administration of adapalene to a patient in whom the inflammatory acneic lesions underexpress CD1d.

The present invention also features administration of adapalene, or of a pharmaceutically acceptable salt thereof, for decreasing the intra-keratinocyte production of IL-10.

In particular, this invention features administration of adapalene to a patient in whom the cells of the acneic epidermis overproduce IL-10.

The patients targeted are preferably patients in whom the inflammatory acneic lesions underexpress CD1d and/or patients in whom the cells of the acneic epidermis, in particular the keratinocytes, overproduce IL-10.

This invention also features the formulation of adapalene into medicaments useful for modulating the immune response of the epidermis by increasing the expression of CD1d and/or by decreasing the expression of IL-10 by the cells of the acneic epidermis, in particular the keratinocytes.

For the treatments described above, the medicament is preferably applied topically and may be in the form of a gel, a lotion or a cream. The present invention therefore features pharmaceutical compositions comprising, formulated into a physiologically acceptable medium, a thus effective amount of adapalene or a pharmaceutically acceptable salt thereof.

This invention therefore also features a regime or regimen for inducing an increase in the expression of IL-10 and/or decreasing the expression of CD1d by the cells of the epidermis of a patient requiring treatment, wherein adapalene or a pharmaceutically acceptable salt thereof is administered to said patient.

The present invention will now be more fully described in the detailed description which follows.

### BRIEF DESCRIPTION OF THE DRAWINGS

FIGS. 1(a) and 1(b) indicate the expression of CD1d in explants of healthy human skin (a) and in explants of acneic skin (b), and

FIGS. 2(a) and 2(b) indicate the expression of IL-10 in explants of healthy human skin (a) and in explants of acneic skin (b).

### DETAILED DESCRIPTION OF BEST MODE AND SPECIFIC/PREFERRED EMBODIMENTS OF THE INVENTION

Adapalene is a chemically stable derivative of naphthoic acid. The name of this derivative is the following: 6-[3-(1-adamantyl)-4-methoxyphenyl]-2-naphthoic acid. A method for preparing this compound is described in EP358574.

Adapalene is a second-generation topical retinoid (like tazarotene) which binds selectively to the RARγ (found mainly in the epidermis) and RARβ (found mainly in the fibroblasts of the dermis) subtypes of the nuclear retinoic acid receptor (RAR), activating the genes responsible for cell differentiation. It does not, on the other hand, bind to the cellular retinoic acid binding proteins. However, clinically, adapalene also exhibits an activity on superficial inflammatory lesions (Millikan L E., Int. J. Dermatol., 2000: 39: 784-788). The mechanisms of the anti-inflammatory activity appear in particular to be related to the modulation of nonspecific immunity. Thus, adapalene inhibits the oxidative metabolism of arachidonic acid via the lipoxygenase pathway, the chemotaxis of polymorphonuclear neutrophils and the production of free radicals. It also inhibits the production of leukotrienes via the lipoxygenase pathway.

Moreover, in the context of the present invention, two molecules have been identified, expressed by the cells of the acneic epidermis, which play an essential role in the development of cutaneous inflammation: CD1d and IL-10.

CD1d:

CD1d belongs to a conserved group of nonpolymorphic cell surface glycoproteins which play the role of antigen-presenting molecules. These glycoproteins are structurally, and in terms of distance, related to the proteins of the conventional major histocompatibility complex class I. CD1d molecules are capable of presenting lipid and glycolipid antigens to NKT cells. CD1d expression is rapidly induced on the keratinocytes of normal skin when the latter undergoes a physical trauma which destroys its barrier function (Aderem A. et al., Nature 2000: 406: 782-787). This shows that the keratinocytes are capable of functioning as unconventional antigen-presenting cells and of activating the NKT cells by presenting lipid antigens by means of CD1d.

IL-10:

Human IL-10 is a 19 kDa lymphokine produced by various cell types, such as B and T cells and monocytes. In normal human skin, this cytokine is not expressed in the keratinocytes, but expression thereof can be induced by UV-B irradiation or by antigenic stimulation. In vitro, normal human keratinocytes express the mRNA encoding IL-10. IL-10 expression is increased after stimulation with alpha-MSH. This cytokine exerts immunosuppressive and anti-inflammatory effects, produced in particular via an inhibitory effect on the production of gamma-interferon (Suh D H et al., Eur. J. Dermatol., 2002: 12: 139-144).

In the context of the present invention, it has been shown that adapalene induces a decrease in or significantly decreases the expression of CD1d by the cells of acneic epidermis, in particular the keratinocytes, thus increasing their function as antigen-presenting cells with respect to NKT cells. The antigens concerned are in particular the lipid, glycolipid and bacterial antigens present in the epidermis or the follicle, and among them, P. acnes antigens.

It has also now been demonstrated that adapalene induces a decrease in or significantly decreases the expression of IL-10 in the epidermis of normal skin and in acne lesions, thereby facilitating the activation of T lymphocytes by increasing interactions from Langerhans cells and T lymphocytes.

These modulations, and in particular the decrease in expression, make it possible to increase the interactions from dendritic cells and T lymphocytes and reinforce the antimicrobial activity against P. acnes.

The term “modulation” means an effect on the expression or the activity of the IL-10 and/or CD1d gene or of the respective promoter thereof, or alternatively on the expression product of these genes.

The term “expression product of the gene” means proteins or any product resulting from the transcription and/or translation of said gene.

The term “activity” means biological activity. The expression “activity of the gene promoter” means the ability of the promoter to induce transcription of the DNA sequence placed under the control of said promoter.

The present invention therefore features formulation of adapalene into compositions for modulating the immune response of the epidermis by increasing the expression of CD1d and/or by decreasing the expression of IL-10 by the cells of acneic epidermis, in particular the keratinocytes. In fact, as indicated in the examples below, under the action of adapalene, the expression of CD1d is significantly increased in the cells of the epidermis, and in particular in the keratinocytes, thus increasing their function as antigen-presenting cells. Similarly, since IL-10 is an immunosuppressive cytokine which has a role in inhibiting the activation and proliferation of T cells, the decrease in this cytokine, induced by adapalene, makes it possible to increase immune responses.

One particular embodiment of the invention features formulation of adapalene, or of a pharmaceutically acceptable salt thereof, into pharmaceutical compositions useful for increasing the expression of CD1d in a skin lesion, in particular in an inflammatory acneic lesion.

The term “increase” means a rise, an overexpression or an elevation in the level of expression of the CD1d gene or an overproduction of the expression product thereof (mRNA, protein).

Another particular embodiment of the invention features formulation of adapalene, or of a pharmaceutically acceptable salt thereof, into pharmaceutical compositions useful for decreasing the intra-keratinocyte production of IL-10.

The term “decrease” means a fall, a reduction, a negative regulation, an inhibition or a suppression (total or partial) of the expression of the IL-10 gene or of the expression product thereof (mRNA, protein).

The term “partial inhibition” means a reduction of at least 25% in the activity, and preferably of 35%, even more preferably of at least 50%.

Preferably, the subject pharmaceutical compositions are useful for treating acne, optimally, in patients in whom the inflammatory acneic lesions underexpress CD1d and/or in patients in whom the cells of the acneic epidermis, in particular the keratinocytes, overproduce IL-10.

The pharmaceutical compositions comprise adapalene or a pharmaceutically acceptable salt thereof, formulated into a physiologically acceptable medium.

To provide an order of magnitude, the compositions according to the invention advantageously comprise from 0.001% to 5% to advantageously from 0.01% to 1% by weight of adapalene, relative to the total weight of the composition, preferably from 0.1% to 0.4% by weight of adapalene, even more preferably 0.3% by weight of adapalene.

Such compositions may also comprise any additive normally employed in the cosmetics or pharmaceutical field, such as propenetrating agents, wetting liquid surfactants, sequestering agents, antioxidants, sunscreens, preservatives, fillers, electrolytes, humectants, dyes, usual inorganic or organic bases or acids, fragrances, essential oils, cosmetic active agents, moisturizing agents, vitamins, essential fatty acids, sphingolipids, self-tanning compounds such as DHA, and agents for soothing and protecting the skin, such as allantoin. Of course, those skilled in the art will take care to select this or these optional additional compound(s), and/or the amount thereof, in such manner that the advantageous properties of the compositions according to the invention are not, or are not substantially, impaired.

These additives may be present in the composition in a proportion of from 0% to 20% by weight relative to the total weight of the composition.

Such a composition may be administered by any known route, conventionally associated with the treatment (administering adapalene) of a dermatological disorder, i.e., topically, enterally, parenterally or ocularly.

Preferably, the treatment is administered topically.

The pharmaceutical composition is preferably in the form of a gel, a lotion or a cream.

Conventionally, the composition is in the form of an aqueous gel. The term “aqueous gel” means a composition containing, in an aqueous phase, a viscoelastic mass formed from colloidal suspensions (gelling agent).

Exemplary gelling agents include those of the polyacrylamide group, such as the sodium acryloyldimethyltaurate copolymer/isohexadecane/polysorbate 80 mixture marketed under the trademark Simulgel 600 by Seppic, the polyacrylamide/isoparaffin C13-14/laureth-7 mixture such as, for example, that marketed under the trademark Sepigel 305 by Seppic, the group of acrylic polymers coupled to hydrophobic chains, such as the PEG-150/decyl/SMDI copolymer marketed under the trademark Aculyn 44 (polycondensate comprising at least, as elements, a polyethylene glycol comprising 150 or 180 mol of ethylene oxide, decyl alcohol and methylenebis(4-cyclohexylisocyanate) (SMDI), at 35% by weight in a mixture of propylene glycol (39%) and water (26%)), the group of modified starches, such as the modified potato starch marketed under the trademark Structure Solanace, or else mixtures thereof.

The preferred gelling agents are from the polyacrylamide group, such as Simulgel 600 or Sepigel 305, or mixtures thereof.

The gelling agent as described above can be included at the preferred concentrations ranging from 0.1% to 15%, and more preferably ranging from 0.5% to 5%.

The compositions are useful for the treatment of inflammatory lesions of any type of acne, i.e., in particular, acne vulgaris, comedonal acne, polymorphous acne, acne rosacea, nodulocystic acne, acne conglobata, senile acne, and secondary acne such as solar acne, acne medicamentosa, work-related acne or occupational acne.

### FIGURES OF DRAWINGS

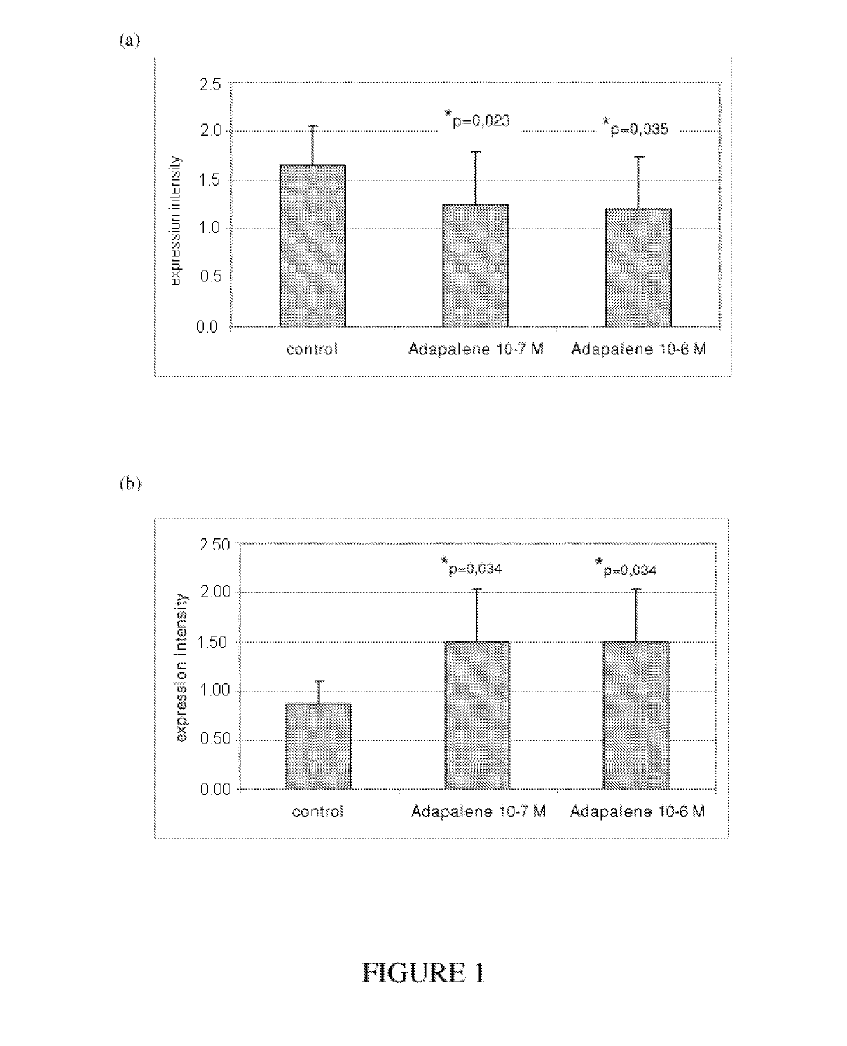

FIG. 1: Expression of CD1d in explants of healthy human skin (a) and in explants of acneic skin (b) after incubation for 24 hours with 10⁻⁷ M and 10⁻⁶ M of adapalene or of control medium (n=8 in each group).

Bars and error bars represent mean and SEM.*p<0.05 [adapalene versus control paired Wilcoxon test].

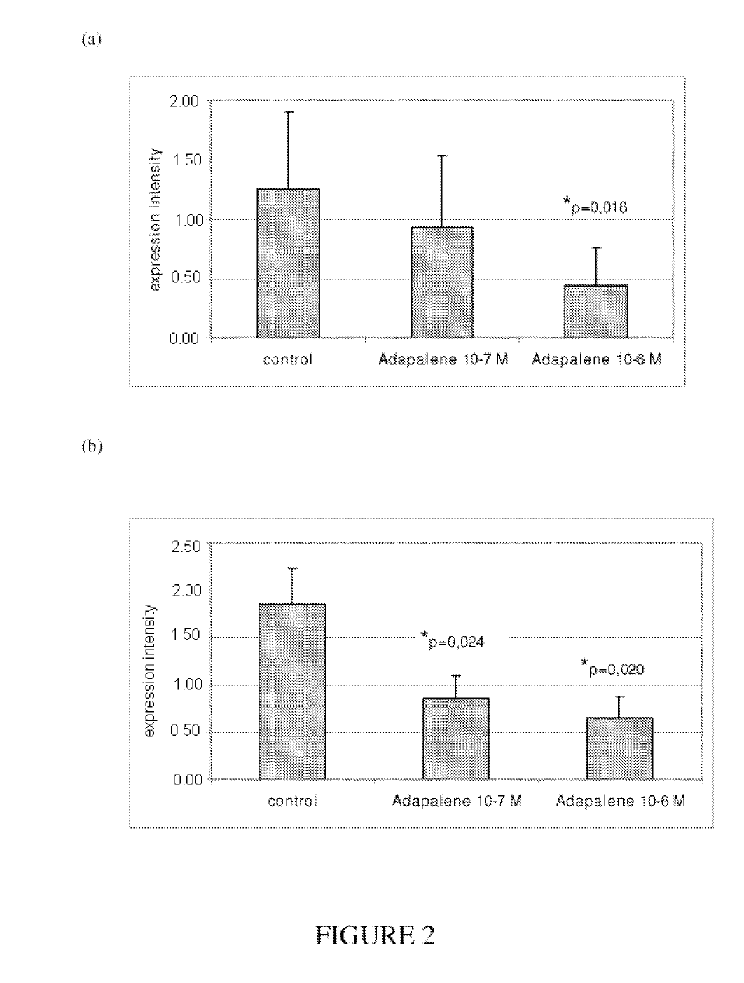

FIG. 2: Expression of IL-10 in explants of healthy human skin (a) and in explants of acneic skin (b) after incubation for 24 hours with 10⁻⁷ M and 10⁻⁶ M of adapalene or of control medium (n=8 in each group).

Bars and error bars represent mean and SEM.*p<0.05 [adapalene versus control paired Wilcoxon test].

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

Skin Biopsies:

Inflammatory lesions of acne: a skin biopsy (1×1.5 cm) was obtained under local anaesthesia from a superficial inflammatory lesion (papula) on the back in 8 patients suffering from moderate acne and undergoing no topical treatment (for the last two weeks) or general treatment (for the last month, or three months for isotretinoin). The fragments taken were immediately placed in culture.

Healthy human skin: fragments of healthy skin originating from 8 healthy donors were obtained from a mammary plasty (plastic surgery) or a foreskin (infant surgery).

Sampling Technique:

Fragments (4 mm in diameter) originating from biopsies of inflammatory skin, considered to be an inflammatory model, and of healthy skin, were incubated at 37° C. in a humid atmosphere and in the presence of 5% CO₂ for 24 hours in KGM culture medium without hydrocortisone (PromoCell GmbH, Heidelberg, Germany). The culture medium was supplemented with two different concentrations of adapalene (Galderma, Sophia Antipolis, France): 10⁻⁷ M and 10⁻⁶ M. The culture medium alone served as control medium.

After incubation, the explants were removed from the culture medium and were then frozen in liquid nitrogen for the immunohistochemical study. The culture supernatants were stored at −80° C. for subsequent measurement of protein secretion, by an ELISA assay technique.

Antibodies Used for the Immunohistochemistry:

The primary antibodies [an anti-human IL-10 mouse monoclonal IgG1 (Diaclone, Besançon, France), an anti-CD1d rabbit polyclonal antibody (Santa Cruz Biotechnology Inc.)] were used, in immunohistochemistry, at the concentration of 2 μg/ml (anti-IL-10 antibody) or 5 μg/ml (anti-CD1d antibody). A negative control mouse IgG1 used at 2 μg/ml (DAKO, Copenhagen, Denmark) or the secondary antibody alone were used as controls.

Immunohistochemistry on Skin Sections:

Sections of frozen samples (5 μm thick) were cut on a cryostat, and were then fixed with acetone at 4° C. for 10 minutes. After saturation of the nonspecific sites with TBS (tris-buffered saline)/BSA (bovine serum albumin) at 0.1%, for 30 minutes at ambient temperature, the slides were incubated for 30 minutes with the primary antibodies at ambient temperature, in a humid atmosphere and in the dark. The sections were then again washed for 15 min in TBS/0.1% BSA/0.05% Tween 20, and then incubated with a biotinylated secondary antibody (DAKO) for 30 min, at ambient temperature and in a humid atmosphere, and then washed and incubated with streptavidin/peroxidase (DAKO) for 30 min, at ambient temperature and in a humid atmosphere. After a final wash, the reaction products were visualized using the peroxidase substrate AEC (hydrogen peroxide/aminoethylcarbazole) (DAKO), for 5 min. The slides were then counterstained with Mayer's hemalun and observed under a Leitz microscope.

The labeling intensity was quantified according to a semi-quantitative scale: none (0), weak (1), medium (2) and strong (3). For each slide, the labeling intensity was determined using a mean of three fields. In addition, for each cytokine or receptor studied, a mean was established on 8 healthy donors or 8 acneic patients in each of the “control”, “10⁻⁷ M adapalene” and “10⁻⁶ M adapalene” groups. The reading was performed by two independent investigators.

Tests with Cytokine:

The concentrations of IL-10 were measured in the culture supernatants using a solid-phase ELISA (Enzyme Linked ImmunoSorbent Assay) sandwich kit, obtained from BioSource (International Inc, Ca, USA).

The slides were read at 450 nm using an Emax reader (Molecular Devices, CA, USA). The sensitivity of the IL-10 test was less than 1 pg/ml and the minimum detectable level of IL-10 was 7.8 pg/ml.

For each cytokine studied, a mean was established on 8 healthy donors or 8 acneic patients in each of the “control”, “10⁻⁷ M adapalene” and “10⁻⁶ M adapalene” groups.

Statistical Analysis:

All the results are expressed as mean +/−SEM. The statistical significance of differences from the values (by comparison to the control medium) was determined by means of the paired Wilcoxon test. A difference from the values was considered to be statistically significant for p<0.05.

Results:

Expression of CD1d:

Explants of healthy human skin: the expression of CD1d is weak to moderate (1.7+/−0.4) in all the healthy human skin epidermises incubated in control medium. Adapalene significantly decreases the expression of CD1d in the epidermis of the healthy donors by comparison with the control medium (p=0.023 with 10⁻⁷ M of adapalene, p=0.035 with 10⁻⁶ M of adapalene) (FIG. 1a).

Explants of acneic skin: a significantly weaker expression (0.9+/−0.2) of CD1d is detected in all the epidermises of acne biopsies by comparison with the healthy skin (p=0.002). The expression of CD1d is significantly increased with the two adapalene concentrations, ranging from 0.9 (+/−0.2) in the control medium to 1.5 (+/−0.5) (p=0.034), in the presence of 10⁻⁷ and 10⁻⁶ M of adapalene (FIG. 1b).

Expression of IL-10:

Explants of healthy human skin: the mean expression of the cytokine is weak in the epidermis incubated with a control medium (1.3 +/−0.7). IL-10 is mainly expressed via the cytokines of the basal layer of the epidermis. The addition of adapalene significantly decreases the expression of IL-10 in a dose-dependent manner. The inhibitory effect of 10⁻⁶ M of adapalene on the expression of IL-10 is significant by comparison with the control medium (p=0.016) (FIG. 2a).

Explants of acneic skin: the expression of the cytokine is medium (1.9 +/−0.4) in the basal layer of the acneic epidermis incubated with the control medium. It is slightly stronger in the acneic epidermis (1.9+/−0.4) than in the healthy epidermis (1.3+/−0.7). The expression of IL-10 significantly decreases in a dose-dependent manner in the presence of adapalene. Thus, the intensity of IL-10 expression decreases from 1.9 (+/−0.4) to 0.9 (+/−0.2) with 10⁻⁷ M of adapalene (p=0.024) and to 0.6 (+/−0.2) with 10⁻⁶ M of adapalene (p=0.0199) (FIG. 2b).

Secretion of IL-10 in the Culture Supernatants:

To determine whether adapalene affects the secretion of IL-10, the cytokine levels were measured in the explant supernatants.

Healthy human skin: the mean level of IL-10 in the supernatants of healthy human skin explants incubated with the control medium was 17.8 pg/ml (+/−20.9). The addition of 10⁻⁷ M of adapalene increases the secretion of IL-10. No modulation is observed with 10⁻⁶ M of adapalene.

Explants of acneic skin: the mean level of IL-10 in the supernatants of skin explants originating from acneic patients, incubated with the control medium, is 5.7 pg/ml (+/−7.1) less than that of the healthy skin supernatants. There is no modulation of the secretion of IL-10 with adapalene. Specifically, the IL-10 levels were 6.1 pg/ml (+/−7.2) and 4.0 pg/ml with 10⁻⁷ M and 10⁻⁶ M of adapalene, respectively.

The present invention confirms firstly that CD1 d is expressed by the keratinocytes of healthy human skin originating from a region of the body other than the scalp (Aderem A. et al., Nature 2000: 406: 782-787). The results given in the present invention show a high level of expression in the superficial keratinocytes located in proximity to the lipid-rich stratum corneum. CD1d expression was demonstrated in the other layers of epidermis, including the basal keratinocytes (Kawai K et al., J. Dermatol. Sci., 2002: 30: 185-194). These results indicate that the basal and suprabasal keratinocytes are presenting cells involved in glycolipid antigen binding to activate NKT cells (Chatelain R et al., M. Arch. Dermatol. Res., 1998: 290: 477-482). The expression of CD1d is slightly decreased in inflammatory acne epidermis, and adapalene induces a significant increase in the expression of CD1d by the cells of the acneic epidermis, in particular the keratinocytes, thus increasing their function as antigen-presenting cells, for lipid and glycolipid antigens, with respect to NKT cells. In fact, in the skin, keratinocytes can operate as nonprofessional antigen-presenting cells (Koreck A et al., Dermatology 2003: 206: 96-105). The presentation of a lipid antigen, in the skin, by cells expressing CD1d is of primary importance in the host's defense against pathogens. By modulating, preferably by increasing, the expression of CD1d by the cells of acneic epidermis, in particular the keratinocytes, adapalene stimulates their NK activity against the lipid and/or bacterial antigens present in the epidermis or the follicle, P. acnes being among them.

The level of IL-10 expression is not significantly different in healthy skin epidermis and acne lesions. The present invention shows that adapalene significantly decreases the expression of IL-10 in the epidermis of normal skin and in acne lesions, thereby facilitating the activation of T lymphocytes by increasing interactions from Langerhans cells and T lymphocytes. In fact, IL-10 is an immunosuppressive cytokine which inhibits the activation and the proliferation of T cells via the inhibition of antigen-presenting cells, including Langerhans cells (Moore K W et al., Annu. Rev. Immunol., 2001: 19: 683-765). The inhibition of ICAM-1 and of other secondary molecules by IL-10 appears to be an important mechanism which inhibits the antigen-presenting function of Langerhans cells (Chatelain R et al., M. Arch. Dermatol. Res., 1998: 290: 477-482). Thus, on the basis of the known effects of IL-10 on inflammatory responses, decreasing its expression with adapalene makes it possible to increase both the innate immune response and the acquired immune response.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for increasing the expression of CD1d in inflammatory acneic lesions afflicting an individual, comprising administering to such individual a thus effective amount of adapalene or pharmaceutically acceptable salt thereof.

2. The regime or regimen as defined by claim 1, said individual being one in whom the inflammatory acneic lesions underexpress CD1d.

3. A regime or regimen for decreasing the intra-keratinocyte production of IL-10 in an individual in need of such treatment, comprising administering to such individual a thus effective amount of adapalene or pharmaceutically acceptable salt thereof.

4. The regime or regimen as defined by claim 3, said individual being one in whom the cells of the acneic epidermis overproduce IL-10.

5. The regime or regimen as defined by claim 1, said individual being one in whom the inflammatory acneic lesions underexpress CD1d and/or in whom the cells of the acneic epidermis overproduce IL-10.

6. A regime or regimen for the treatment of the inflammatory lesions of acne vulgaris, comedonal acne, polymorphous acne, acne rosacea, nodulocystic acne, acne conglobata, senile acne, secondary acne, solar acne, acne medicamentosa, work-related acne and/or occupational acne, comprising administering to an individual in need of such treatment, a thus effective amount of adapalene or pharmaceutically acceptable salt thereof.

7. A regime or regimen for modulating the immune response of the epidermis by increasing the expression of CD1d and/or by decreasing the expression of IL-10 by the cells of the acneic epidermis, comprising administering to an individual in need of such treatment, a thus effective amount of adapalene or pharmaceutically acceptable salt thereof.

8. The regime or regimen as defined by claim 7, said cells of the acneic epidermis comprising keratinocytes.

9. The regime or regimen as defined by claim 1, comprising topically applying such effective amount of adapalene, formulated into a physiologically acceptable medium therefor, onto said inflammatory acneic lesions afflicting such individual.

10. The regime or regimen as defined by claim 3, comprising topically applying such effective amount of adapalene, formulated into a physiologically acceptable medium therefor, onto the skin of such individual.

11. The regime or regimen as defined by claim 9, said adapalene or salt formulation thereof comprising a gel, a lotion or a cream.

12. The regime or regimen as defined by claim 10, said adapalene or salt formulation thereof comprising a gel, a lotion or a cream.

13. A regime or regimen for inducing an increase in the expression of IL-10 and/or decreasing the expression of CD1d by the cells of the epidermis of an individual in need of such treatment, comprising administering to such individual a thus effective amount of adapalene or pharmaceutically acceptable salt thereof.
